(19) United States
(12) Patent Application Publication
(10) Pub. No.: US 2016/0053219 A1
(43) Pub. Date: Feb. 25, 2016

(54) SYSTEMS, METHODS, AND DEVICES FOR TEMPERATURE CONTROL

(71) Applicant: OpGen, Inc., Gaithersburg, MD (US)

(72) Inventors: George Terrance Walker, Chevy Chase, MD (US); Tony Rockweiler, Arlington, VA (US)

(21) Appl. No.: 14/835,227

(22) Filed: Aug. 25, 2015

Related U.S. Application Data
(60) Provisional application No. 62/041,405, filed on Aug. 25, 2014.

(51) Int. Cl. C12M 1/34; C12M 1/24; C12N 1/04

(52) U.S. Cl. CPC C12M 41/12; C12N 1/04; C12M 23/08

(57) ABSTRACT

Systems, methods, and devices are disclosed for improved temperature control, particularly within a predetermined range of temperatures near or above varying ambient temperatures. Previously-unrealized advantages are recognized for maintaining samples, particularly medical and/or biological specimens, at a temperature within a predetermined range of temperatures near or above ambient temperature that selectively promote and/or selectively inhibit organism growth, organism viability, biochemical reactions, and/or chemical reactions. Systems, methods, and devices may include at least phase change material selected and configured to maintain a sample at a predetermined temperature range between about 22° Celsius and about 100° Celsius during a predetermined time period.

### CROSS-REFERENCE TO RELATED APPLICATIONS

This application claims a priority benefit of U.S. Provisional Patent Application No. 62/041,405, filed on Aug. 25, 2014, and entitled “Systems, Methods, and Devices for Temperature Control,” which application is incorporated herein by reference in its entirety.

### TECHNICAL FIELD

The present disclosure relates generally to systems, methods, and devices for temperature control, particularly maintaining a sample at a temperature within a predetermined range of temperatures. More specifically, the present disclosure relates to systems, methods, and devices for controlled temperature to selectively promote organism growth, organism viability, biochemical reactions, and/or chemical reactions in organic and/or inorganic samples, including medical and/or biological specimens and/or specimen cultures during storage and transport for analytic, diagnostic, therapeutic, and/or monitoring purposes.

### BACKGROUND

Organic and/or inorganic samples, including medical and/or biological specimens may be collected, extracted, and/or prepared for various purposes, including analysis, diagnosis, therapy, and monitoring of disease. For example, some samples may be collected with a swab (e.g., buccal or anal) and wiped across an agar plate, where, for example, bacteria from the swab may grow as a microbiological culture. Medical specimens may also be sampled using, among other techniques, biopsy, venipuncture, fingerstick or fingerprick, urination, stool test, etc., and then collected, with or without culture media and/or inoculation, in vials, plates, Petri dishes, cartridges, or other appropriately-sized containers, which may be labeled and securely sealed to avoid contamination and/or infection.

Organic and/or inorganic samples are often collected in field studies and must be transported to another site (referred to herein as a “laboratory”), such as a microbiology, chemistry, and/or cytology laboratory, for further processing, testing, and/or analysis. For example, after sampling and/or collection, medical specimens are often transported to a laboratory, such as a medical or clinical laboratory.

### SUMMARY

For best results, samples, particularly medical specimens, should be transported to a laboratory as soon as possible after collection and not be exposed to extreme cold, excessive heat, or too much sunlight during transport. However, the inventors have recognized and appreciated that conditions for transporting samples can vary widely, depending on factors including, but not limited to, travel distance/time, geographic location, season, etc.

Delay can be a costly side-effect of storing and/or transporting samples. Once a laboratory receives a sample, particularly a microbiological specimen, further time-consuming processing and extraction techniques may be required. Some tests like microbial identification and antibiotic susceptibility testing can be performed on a microbiological specimen without much further processing, but many analytic, diagnostic, therapeutic, and/or disease monitoring tests (e.g., molecular genotyping assay analysis) require selective amplification of target microorganisms (and/or their DNA). Importantly, microorganisms/DNA have the disadvantage of low sensitivity (i.e., high detection limits) for molecular genotyping assay analysis. Therefore, laboratories employ techniques (e.g., micro plate cultures) to select and amplify target microorganisms (and thus the level of target DNA) in culture, thus increasing test sensitivity. However, like transporting samples, these techniques can be time consuming. Of course, depending on laboratory demand and supply, samples may even need to be stored as part of a backlog until laboratory resources are available to proceed with further processing and analysis. It is particularly important to minimize or find a way to utilize these delays when a specimen is being tested to analyze, diagnose, treat, and/or monitor multi-drug-resistant microorganisms before infection spreads in a patient or population.

The inventors recognized and appreciated that one challenge for effectively and efficiently storing and/or transporting samples is thermal protection, particularly from unwanted heat absorption. Thermal protection substances (e.g., ice/water) have been used primarily to maintain samples at reduced temperatures that inhibit all microbiological growth and/or chemical activity. However, this disclosure recognizes previously-unrealized advantages to maintaining a sample at a temperature within a predetermined range of temperatures near or above ambient temperature that do not inhibit or at least only selectively inhibit microbiological growth and/or chemical activity.

In applications other than storage and transport of samples, some amount of heat is maintained in an object/space for a desired time period by supplying either insulation to prevent heat dissipation or a thermal energy source to resupply heat using conduction, radiation, convection, etc. Insulation merely slows the dissipation of heat and, depending on factors such as surface area, may not maintain enough heat for the duration of a desired time period. Likewise, heated materials with high specific heat capacity (e.g., hot water bottles) are time-limited. Meanwhile, electric incubators or heating apparatuses are not limited by time, but do require constant or at least near-constant supply of electricity to maintain a heat supply. Finally, chemical heat sources, such as disposable chemical pads, are typically limited to a one-time exothermic chemical reaction. The most common chemical pads generate heat by flexing a small flat disc of notched ferrous metal embedded in a supersaturated solution (of, e.g., sodium acetate in water) to trigger exothermic crystallization of the solution into a hydrated salt (e.g., sodium acetate trihydrate). Because chemical pads and other heat sources employ non-equilibrium processes, their heat supply is not only time-limited but also non-adjustable.

Thus, a need remains for systems, methods, and devices for improved temperature control, particularly for maintaining a temperature within a predetermined range of temperatures near or above ambient temperature. More specifically, a need remains for improved systems, methods, and devices for maintaining the temperature of samples, particularly medical and/or biological specimens, so as to effectively and more efficiently promote selective organism growth, organism viability, biochemical reactions, and/or chemical reactions during storage and/or transport.

The present disclosure provides systems, methods, and devices for improved temperature control, particularly for maintaining a temperature within a predetermined range of temperatures near or above ambient temperature. In particular, the inventors have recognized previously-unrealized advantages to maintaining samples, particularly medical and/or biological specimens, at a temperature within predetermined ranges of temperatures near or above ambient temperature that do not inhibit or at least only selectively inhibit microbiological growth and/or chemical activity. Thus, improved systems, methods, and devices are disclosed for maintaining the temperature of samples so as to effectively and more efficiently use storage and/or transport time to selectively promote organism growth, organism viability, biochemical reactions, and/or chemical reactions. By maintaining a temperature of a sample within a predetermined range of temperatures, some embodiments avoid or at least counteract additional delay following receipt and/or storage by a laboratory.

According to one embodiment, a system includes at least one pack having sealed therein at least one phase change material selected and configured to maintain a predetermined temperature range between about 22° Celsius and about 100° Celsius during a predetermined time period, and a structure configured to receive and retain the at least one pack. In an embodiment, the system maintains at least one sample within the predetermined temperature range during the predetermined time period. The structure may be further configured to receive and retain the at least one sample. The system may promote organism growth, organism viability, a biochemical reaction, and/or a chemical reaction in the at least one sample during the predetermined time period. The system may inhibit organism growth, organism viability, a biochemical reaction, and/or a chemical reaction in the at least one sample during the predetermined time period. The sample may be an inorganic sample or an organic sample. The organic sample may be a biological specimen or a biological specimen culture.

In an embodiment, the system is used to at least one of store and transport the at least one sample. The system may be further configured such that pre-analytical processing, analytical testing, medical diagnostic testing, and/or medical therapy is applied to the at least one sample during the predetermined time period. The predetermined time period may be a storage time period and/or a transport time period. The structure may be insulated. The structure may include polystyrene foam. The at least one pack may be pre-heated to an initial temperature, the initial temperature being approximately the same as a phase change temperature of one of the at least one phase change material. The initial temperature may be the phase change temperature +/2° Celsius. The phase change temperature may be the melting temperature of the one of the at least one phase change material. The initial temperature may be about 40° Celsius.

In an embodiment, the system further includes a monitoring device including at least one sensor. The at least one sensor may be incorporated into the structure and/or the at least one pack. The at least one sensor may be configured to be in contact with at least one sample within the structure. The monitoring device may be adapted to at least one of record and transmit data representative of signals from the at least one sensor. The data representative of signals from the at least one sensor may include at least one of temperature-related data, location-related data, pressure-related data, radiation-related data, and shock-related data. The at least one sensor may include a thermometer, a thermistor, a thermocouple, a global positioning system (GPS) receiver, a global navigation satellite system (GNSS) receiver, a transducer, a radiometer, a dosimeter, and/or an accelerometer.

In an embodiment, the predetermined temperature range may be between about 33° Celsius and about 41° Celsius. The predetermined temperature range may be 37° Celsius +/−2° Celsius. The predetermined time period may be between about 2 hours and about 12 hours, between about 12 hours and about 24 hours, between about 24 hours and about 48 hours, between about 2 days and about 1 week, and/or between about 1 week and about 1 month.

In an embodiment, the at least one phase change material includes a paraffin, a fatty acid, a salt hydrate, a eutectic composition, a cross-linked polyethylene, and/or a polyalcohol. The at least one phase change material may include n-Eicosane. The n-Eicosane may be initially in a substantially liquid phase. The at least one phase change material may include heneicosane. The heneicosane may be initially in a substantially solid phase.

In another embodiment, a system includes a structure including at least one phase change material selected and configured to maintain a predetermined temperature range between about 22° Celsius and about 100° Celsius during a predetermined time period. The at least one phase change material may be encapsulated in at least one compartment integrated into the structure. The at least one phase change material may be a thermal composite integrated into the structure.

In another embodiment, a device for promoting and/or inhibiting organism growth, organism viability, a biochemical reaction, and/or a chemical reaction in at least one sample includes at least one phase change material encapsulated by an inert material, the inert material being selected and configured to receive and retain at least one sample, the at least one phase change material being selected and configured to maintain the at least one sample within a predetermined temperature range between about 22° Celsius and about 100° Celsius during a predetermined time period, such that organism growth, organism viability, a biochemical reaction, and/or a chemical reaction is promoted in the at least one sample during the predetermined time period. The at least one sample may be retained in culture media. The culture media may include at least one antibiotic for selection of Gram-negative carbapenamase resistant enterobacteriacae (CRE). The culture media may include at least one Gram-positive bacterium inhibitor for selection of at least one of Gram negative-CRE and Extended Spectrum Beta-lactamase (ESBL) bacteria. The at least one Gram-positive bacterium inhibitor may be a pH indicator and/or a bile salt.

In an embodiment, a pack for promoting and/or inhibiting organism growth, organism viability, a biochemical reaction, and a chemical reaction in at least one sample during storage and/or transport includes a container with at least one internal compartment having sealed therein at least one phase change material selected and configured to maintain the at least one sample within a predetermined temperature range between about 22° Celsius and about 100° Celsius during a predetermined time period, such that organism growth, organism viability, a biochemical reaction, and/or a chemical reaction is promoted in the at least one sample during the predetermined time period. The container may include at least one first internal compartment and at least one second internal compartment. The at least one first internal compartment may have n-Eicosane sealed therein, and the at least one second internal compartment may have heneicosane sealed therein.

In an embodiment, a method for controlling temperature includes sealing, within a structure, at least one pack having sealed therein at least one phase change material selected and configured to maintain a predetermined temperature range between about 22° Celsius and about 100° Celsius during a predetermined time period, and maintaining the predetermined temperature range during the predetermined time period.

In an embodiment, a method is disclosed for using at least one phase change material to maintain at least one sample within a predetermined temperature range higher than ambient temperature during a predetermined time period, wherein the at least one phase change material is selected and configured to promote at least one of organism growth, organism viability, a biochemical reaction, and a chemical reaction in the at least one sample. The at least one phase change material may be selected and configured to promote microbiological organism growth. The predetermined time period may be an estimated time period for storing and/or transporting the at least one sample.

In an embodiment, a method is disclosed for using at least one phase change material to maintain at least one sample within a predetermined temperature range lower than ambient temperature during a predetermined time period, wherein the at least one phase change material is selected and configured to promote organism growth, organism viability, a biochemical reaction, and/or a chemical reaction in the at least one sample. The at least one phase change material may be selected and configured to promote microbiological organism growth. The predetermined time period may be an estimated time period for storing and/or transporting the at least one sample.

In an embodiment, a method is disclosed for using at least one phase change material to maintain at least one sample within a predetermined temperature range between about 22° Celsius and about 100° Celsius during a predetermined time period, wherein the at least one phase change material is selected and configured to promote organism growth, organism viability, a biochemical reaction, and/or a chemical reaction in the at least one sample method of using at least one phase change material to maintain a temperature of at least one sample.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Other systems, processes, and features will become apparent to those skilled in the art upon examination of the following drawings and detailed description. It is intended that all such additional systems, processes, and features be included within this description, be within the scope of the present invention, and be protected by the accompanying claims.

### BRIEF DESCRIPTION OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 is a photograph illustrating a system or kit for storing and/or transporting samples in accordance with some embodiments.

FIGS. 2-6 are flow diagrams illustrating processes for collecting and transporting samples for further testing and analysis in accordance with some embodiments.

FIG. 7 is a plot of temperature inside a carrier for storing and/or transporting samples as a function of time in accordance with some embodiments.

FIGS. 8-10 are flow diagrams illustrating processes for incubating specimen culture plates for further testing and analysis in accordance with some embodiments.

FIG. 11 is a series of photographs illustrating specimen culture plates after an incubation period in accordance with some embodiments.

FIG. 12 is a diagram illustrating a carrier for storing and/or transporting a sample in accordance with some embodiments.

FIG. 13 is a plot of temperature inside the carrier of FIG. 12 as a function of time in accordance with some embodiments.

### DETAILED DESCRIPTION

The present disclosure provides systems, methods, and devices incorporating phase control materials for improved temperature control, particularly for maintaining a temperature within a predetermined range of temperatures near or above ambient temperature. More specifically, the present disclosure recognizes previously-unrealized advantages to maintaining organic and/or inorganic samples at a temperature within predetermined ranges of temperatures near or above ambient temperature that do not inhibit or at least only selectively inhibit microbiological growth and/or chemical activity. Thus, improved systems, methods, and devices incorporating phase control materials are disclosed for maintaining the temperature of samples so as to effectively and more efficiently use storage and/or transport time to selectively promote organism growth, organism viability, biochemical reactions, and/or chemical reactions. By maintaining a temperature of a sample within a predetermined range of temperatures, some embodiments avoid or at least counteract additional delay following receipt and/or storage by a laboratory.

One or more phase change materials may be selected and incorporated into the disclosed systems, methods, and devices in sufficient quantities to maintain a temperature within predetermined ranges of temperatures for predetermined time periods. The disclosed systems, methods, and devices may be modified to achieve a particular temperature range and/or time period. The temperature range and/or time period may be selected to selectively promote (and/or inhibit) organism growth, organism viability, biochemical reactions, and/or chemical reactions. For example, one or more phase change materials may be selected in particular quantities to maintain a temperature within a range of temperatures predetermined to selectively promote amplification of a target type of organism (and potentially inhibit another type of organism) in a specimen culture over a time period (e.g., between 2 hours and 1 week) commensurate with and/or necessitated by storage and/or transport of the specimen. As a result, the level of the target type of organisms and/or ratio of target organisms to non-target organism is effectively and efficiently increased without requiring further amplification (and additional delay) following the storage and/or transport of the specimen.

### Phase Change Materials

Phase change materials are sustainably reusable because they reversibly undergo solid/liquid transitions, during which they either absorb or release heat to reach equilibrium. According to some embodiments, phase change materials can be incorporated into insulated or non-insulated containers or packs in order to absorb heat from or release heat to surrounding materials, including organic and/or inorganic samples. While some substances (e.g., ice/water) are commonly used to maintain samples at reduced temperatures that inhibit microbiological growth, phase change materials have not been utilized to release heat to maintain temperatures within ranges near or above ambient temperature (e.g., 22° Celsius to 100° Celsius), particularly temperatures that have been predetermined to selectively promote (and/or inhibit) organism growth, organism viability, biochemical reactions, and/or chemical reactions.

According to some embodiments, one or more phase change materials are incorporated into systems, methods, and devices to effectively and efficiently store and/or transport samples while providing thermal protection, e.g., by absorbing unwanted heat from the environment. Some phase change materials described herein may be selected and used to maintain samples at reduced temperatures that inhibit all microbiological growth and/or chemical activity. However, this disclosure recognizes previously-unrealized advantages to maintaining a sample at a temperature within a predetermined range of temperatures near or above ambient temperature that do not inhibit or at least only selectively inhibit microbiological growth and/or chemical activity. Unlike ice/water and other substances that absorb heat while changing from solid to liquid at higher temperatures, some phase change materials described herein release heat while changing from liquid to solid at lower temperatures. Thus, an object/space can be maintained at a higher temperature for a desired time period using the phase change equilibrium process.

Compared to other thermal energy sources (e.g., materials with high specific heat capacity, electric incubators or heating apparatuses, and chemical reactions), phase change materials provide a simple, robust, and inexpensive way to adjust and maintain samples at or near constant temperature regardless of changes in ambient temperature, e.g., during shipment.

Because phase change materials cycle between solid and liquid phases, encapsulation is preferred. For example, microscopic-sized particles of phase change materials may be coated to form beads and, in some embodiments, suspended within a continuous phase such as water (i.e., a phase change slurry). Alternatively, molecular-encapsulation allows a very high concentration of phase change material within a polymer compound as well as drilling and cutting through the material without the phase change material leaking

Phase change materials may also be combined with other solid structures (porous if the phase change material is required to flow) to form thermal-composite materials, such as copper-mesh immersed in a paraffin wax. The inclusion of thermal composites may increase bulk thermal conductivity by adding a highly conducting solid (e.g., copper-mesh) into a relatively low conducting phase change material (e.g., paraffin wax).

Phase change materials often perform best in small packages. Accordingly, phase change materials may be divided into small packages or cells within larger packages. The packaging material may be selected to conduct heat well, withstand frequent volume changes as phase changes occur, and/or restrict the passage of water, leakage of phase change materials, and/or corrosion. Common packaging materials showing chemical compatibility with room temperature phase change materials include but are not limited to stainless steel, polypropylene, and polyolefin.

Phase change materials may be incorporated into systems, methods, and devices of the present disclosure in different ways including, but not limited to, the following embodiments. For example, one or more phase change materials may be inserted and/or incorporated into a storage and/or transport container. Phase change materials may be integrated with the packaging, for instance, in the walls of the container and/or compartments or other structures formed within the container (e.g., a rack configured to receive sample collection containers). More simply, one or more phase change materials could be sealed in modular packs (e.g., in bottles or zipper storage bags) that can be inserted into a storage and/or transport container.

Alternatively, one or more phase change materials may be inserted and/or incorporated into a sample collection container like a vial (e.g., molded into the typically concave bottom or encapsulated with an inert material as a small bead that can be inserted into the vial and even be magnetic and/or shaped to promote stirring/mixing in the vial). One or more phase change materials also may be incorporated into a sample collection device like a swab (e.g., in the swab shaft) or even embody the sample collection device (e.g., encapsulated with an inert material, such as a small bead that can be, e.g., inserted and then removed from a subject's mouth).

If more than one phase change material is used, the phase change materials may be incorporated into and/or stored in different compartments of the same container, pack, or device, or incorporated into and/or stored in different containers, packs, or devices. For embodiments in which the phase change materials are to be pre-heated, the container(s), pack(s), or device(s) incorporating and/or storing the phase change materials may be designed to be compatible with a heating device, including as part of a larger system.

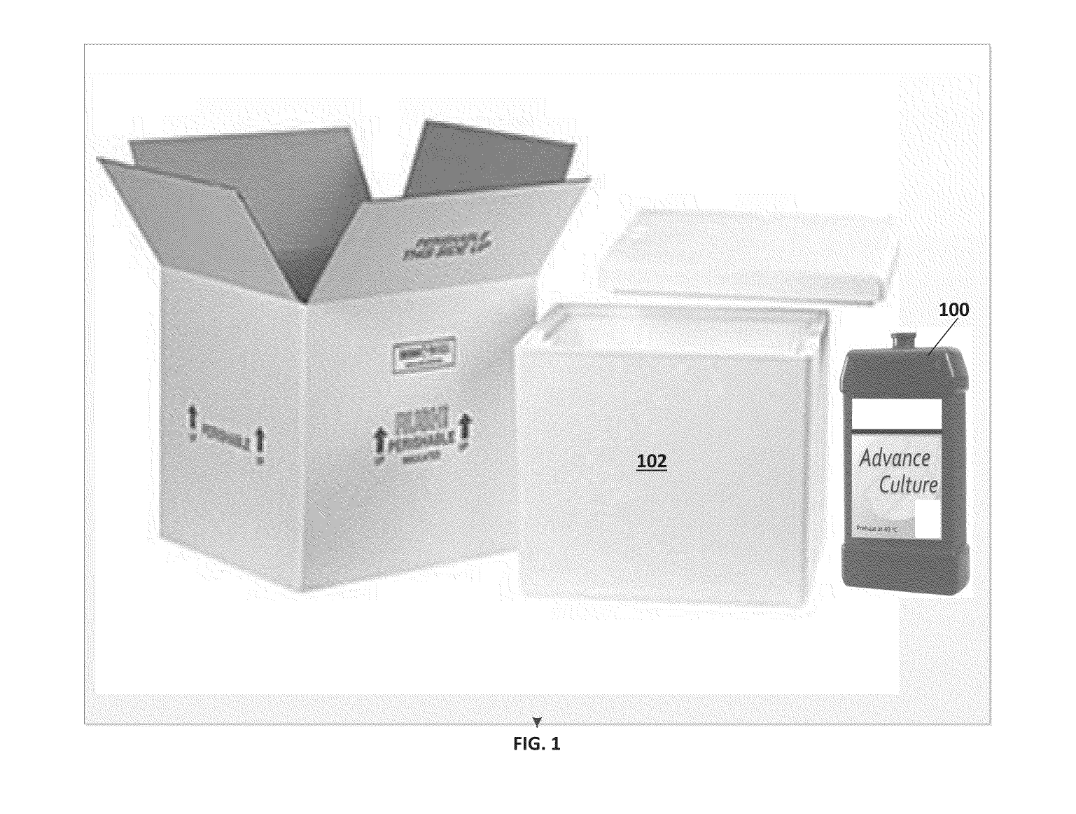

FIG. 1 is a photograph illustrating a system or kit for storing and/or transporting one or more samples in accordance with some embodiments. A phase change material pack 100 is packed into the internal cavity of an insulated carrier 102 (e.g., a Styrofoam™ cooler), which is also configured to hold the samples during shipment. According to some embodiments, insulation is used to further prevent heat absorption and/or dissipation; however, insulation is not always necessary. Likewise, according to some embodiments, one or more phase change materials are pre-heated to a temperature at or near a phase change temperature of the one or more phase change materials; however, pre-heating is not always necessary. A phase change temperature of a phase change material is an approximate temperature at which the phase change material changes phase, e.g., from solid to liquid or vice versa (i.e., the melting temperature of the phase change material is a phase change temperature).

Phase change materials melt at very specific temperatures. For example, n-Eicosane, which is a paraffin, melts at about 37° Celsius. According to some embodiments, when a sample is packed in an insulated carrier (e.g., a Styrofoam™ cooler) along with a container or pack of liquid n-Eicosane heated to about 39° Celsius, the n-Eicosane will begin to solidify and release heat to maintain the sample at about 37° Celsius. Thus, phase change materials can be used to defend samples from cold exposure (e.g., winter transport conditions).

Phase change materials can also be used to protect microorganisms from high ambient temperatures. For example, n-Heneicosane melts at about 39-41° Celsius. According to some embodiments, when a sample is packed in an insulated carrier (e.g., a Styrofoam™ cooler) along with a container or pack of solid n-Heneicosane at about 39° Celsius and the package is exposed to elevated ambient temperatures, the solid n-Heneicosane will absorb heat as it melts and protect the sample from high heat exposure (e.g., summer transport conditions).

More than one type of phase change material may be incorporated in one embodiment. For example, a phase change material that absorbs heat while changing from solid to liquid at a higher temperature (e.g., about 60° Celsius) may be combined with a different phase change material that releases heat while changing from liquid to solid at a lower temperature (e.g., about 40° Celsius). In this way, a temperature may be maintained between the two melting points regardless of fluctuations in ambient temperature. There is a large continuum of different pairs of phase change materials that may be selected based on melting temperature to maintain a temperature within a predetermined range of temperatures that selectively promotes (and/or inhibits) organism growth, organism viability, biochemical reactions, and/or chemical reactions.

For example, to account for variable conditions of mesophilic bacteria (i.e., microorganisms such as some species of bacteria, fungi, and even some archaea that are best active at median temperatures), an embodiment can initially incorporate both liquid n-Eicosane and solid n-Heneicosane at about 39° Celsius to protect from heat loss or heat gain during storage and/or transport. For example, the n-Eicosane and n-Heneicosane may be incorporated into and/or stored in different compartments of the same container, pack, or device, or incorporated into and/or stored in different containers, packs, or devices.

A large number of other phase change materials also melt at specific temperatures in any required temperature range from −5° Celsius up to 190° Celsius, including other organic phase change materials like other paraffins (CₙH_{2n|2}) and fatty acids (CH₃(CH₂)₂ₙCOOH); inorganic phase change materials like salt hydrates (MₙH₂O), eutectic compositions, cross-linked polyethylene, and polyalcohols.

Some non-limiting examples of phase change materials that may be incorporated into some embodiments include sodium sulfate (Na2SO4.10H2O), NaCl.Na2SO4.10H2O, lauric acid, TME(63% w/w)+H2O(37% w/w), Mn(NO3)2.6H2O+MnCl2.4H2O(4% w/w), Na2SiO3.5H2O, Paraffin 14-Carbons, Paraffin 15-Carbons, Paraffin 16-Carbons, Paraffin 17-Carbons, Paraffin 18-Carbons, Paraffin 19-Carbons, Paraffin 20-Carbons, Paraffin 21-Carbons, Paraffin 22-Carbons, Paraffin 23-Carbons, Paraffin 24-Carbons, Paraffin 25-Carbons, Paraffin 26-Carbons, Paraffin 27-Carbons, Paraffin 28-Carbons, Paraffin 29-Carbons, Paraffin 30-Carbons, Paraffin 31-Carbons, Paraffin 32-Carbons, Paraffin 33-Carbons, Paraffin 34-Carbons, formic acid, caprilic acid, glycerin, p-lattic acid, methyl palmitate, camphenilone, docasyl bromide, caprylone, phenol, heptadecanone, 1-cyclohexylooctadecane, 4-heptadacanone, p-joluidine, cyanamide, methyl eicosanate, 3-heptadecanone, 2-heptadecanone, hydrocinnamic acid, cetyl acid, α-nepthylamine, camphene, o-nitroaniline, 9-heptadecanone, thymol, methyl behenate, diphenyl amine, p-dichlorobenzene, oxalate, hypophosphoric acid, o-xylene dichloride, β-chloroacetic acid, chloroacetic acid, nitro naphthalene, trimyristin, heptaudecanoic acid, α-chloroacetic acid, bee wax, glyolic acid, glycolic acid, p-bromophenol, azobenzene, acrylic acid, dinto toluent, phenylacetic acid, thiosinamine, bromcamphor, durene, benzylamine, methyl brombrenzoate, α-napthol, glautaric acid, p-Xylene dichloride, catechol, quinone, acetanilide, succinic anhydride, benzoic acid, stilbene, benzamide, acetic acid, polyethylene glycol 600, capric acid, eladic acid, pentadecanoic acid, tristearin, myristic acid, palmatic acid, stearic acid, acetamide, and methyl fumarate. Thus, depending on the embodiment, other phase change materials or pairs thereof may be selected and incorporated to control temperature to, for example, selectively promote (and/or inhibit) organism growth, organism viability, biochemical reactions, and/or chemical reactions. Table 1, below, lists approximate melting temperatures for some exemplary but non-limiting phase change materials according to some embodiments.

TABLE 1

| | Approximate Melting |
|---|---|
| Phase Change Material | Point (° Celsius) |
| 1-Cyclohexylooctadecane | 41 |
| 2-Heptadecanone | 48 |
| 3-Heptadecanone | 48 |
| 4-Heptadacanone | 41 |
| 9-Heptadecanone | 51 |
| Acetamide | 81 |
| Acetic acid | 16.7 |
| Acrylic acid | 68 |
| Acetanilide | 118.9 |
| α-Chloroacetic acid | 61.2 |
| α-Nepthylamine | 59 |
| α-Napthol | 96 |
| Aluminum | 660.32 |
| Azobenzene | 67.1 |
| Bee wax | 61.8 |
| Benzamide | 127.2 |
| Benzoic acid | 121.7 |
| Benzylamine | 78 |
| β-Chloroacetic acid | 56 |
| Bromcamphor | 77 |
| Camphene | 50 |
| Camphenilone | 39 |
| Capric acid | 36 |
| Caprilic acid | 16.3 |
| Caprylone | 40 |
| Catechol | 104.3 |
| Cetyl acid | 49.3 |
| Chloroacetic acid | 56 |
| Copper | 1,084.62 |
| Cyanamide | 44 |
| Dinto toluent (2,4) | 70 |
| Diphenyl amine | 52.9 |
| Docasyl bromide | 40 |
| Durene | 79.3 |
| Eladic acid | 47 |
| Formic acid | 7.8 |
| Glautaric acid | 97.5 |
| Glycerin | 17.9 |
| Glycolic acid | 63 |
| Glyolic acid | 63 |
| Gold | 1,064.18 |
| Heptadecanone | 41 |
| Heptaudecanoic acid | 60.6 |
| Hydrocinnamic acid | 48 |
| Hypophosphoric acid | 55 |
| Iron | 1,538 |
| KNO3 | 337 |
| KNO3(10%)/NaNO3 | 290 |
| KNO3/KBr(4.7%)/ | 342 |
| KCl(7.3%) | |
| KNO3/KCl(4.5%) | 320 |
| KOH | 360 |
| Lauric acid | 44.2[12] |
| Lead | 327.46 |
| Lithium | 180.54 |
| Methyl brombrenzoate | 81 |
| Methyl behenate | 52 |
| Methyl eicosanate | 45 |
| Methyl fumarate | 102 |
| Methyl palmitate | 29 |
| Mn(NO3)2•6H2O + | 15-25 |
| MnCl2•4H2O(4% w/w) | |
| Myristic acid | 58 |
| Na2SiO3•5H2O | 72.2 |
| NaCl(26.8%)/NaOH | 370 |
| NaCl(42.5%)/KCl(20.5)/ | 385-393 |
| MgCl2 | |
| NaCl(5.0%)/NaNO3 | 282 |
| NaCl(5.7%)/NaNO3 | 287 |
| (85.5%)/Na2SO4 | |
| NaCl/NaNO3 (5.0%) | 284 |
| NaCl/KCL(32.4%)/ | 346 |
| LiCl(32.8%) | |
| NaCl•Na2SO4•10H2O | 18 |
| NaNO2 | 282 |
| NaNO3 | 310 |
| NaOH | 318 |
| NaOH/Na2CO3(7.2%) | 283 |
| Nitro napthalene | 56.7 |
| O-Nitroaniline | 50 |
| O-Xylene dichloride | 55 |
| Oxolate | 54.3 |
| p-Bromophenol | 63.5 |
| p-Dichlorobenzene | 53.1 |
| p-Joluidine | 43.3 |
| p-Lattic acid | 26 |
| p-Xylene dichloride | 100 |
| Palmatic acid | 55 |
| Paraffin 14-Carbons | 5.5 |
| Paraffin 15-Carbons | 10 |
| Paraffin 16-Carbons | 16.7 |
| Paraffin 17-Carbons | 21.7 |
| Paraffin 18-Carbons | 28 |
| Paraffin 19-Carbons | 32 |
| Paraffin 20-Carbons | 36.7 |
| Paraffin 21-Carbons | 40.2 |
| Paraffin 22-Carbons | 44 |
| Paraffin 23-Carbons | 47.5 |
| Paraffin 24-Carbons | 50.6 |
| Paraffin 25-Carbons | 49.4 |
| Paraffin 26-Carbons | 56.3 |
| Paraffin 27-Carbons | 58.8 |
| Paraffin 28-Carbons | 61.6 |
| Paraffin 29-Carbons | 63.4 |
| Paraffin 30-Carbons | 65.4 |
| Paraffin 31-Carbons | 68 |
| Paraffin 32-Carbons | 69.5 |
| Paraffin 33-Carbons | 73.9 |
| Paraffin 34-Carbons | 75.9 |
| Pentadecanoic acid | 52.5 |
| Phenol | 41 |
| Phenylacetic acid | 76.7 |
| Polyethylene glycol 600 | 20 |
| Quinone | 115 |
| Silver | 961.78 |
| Sodium | 32.4 |
| sulfate (Na2SO4•10H2O) | |
| Stearic acid | 69.4 |
| Stibene | 124 |
| Succinic anhydride | 119 |
| Thiosinamine | 77 |
| Thymol | 51.5 |
| Titanium | 1,668 |
| TME(63% w/w) + | 29.8 |
| H2O(37% w/w) | |
| Trimyristin | 33 |
| Tristearin | 56 |
| Water | 0 |
| Zinc | 419.53 |

### Monitoring Devices

According to some embodiments, one or more monitoring systems and/or monitoring devices with one or more sensors may be used with some embodiments to store and/or transmit useful information including, but not limited to, temperature data, location (e.g., GPS) data, pressure data, radiation data, shock data, and other storage or transport conditions. Real-time monitoring can minimize damage and/or loss by allowing for early corrective action, for example, while a sample is in storage or being transported. A monitoring device may include one or more processors adapted for monitoring, for example, temperature levels; memory including, for example, either or both random access memory (RAM) and read-only memory (ROM); programmable logic; a sensor interface; and/or a communications module including, for example, a transceiver I/O, configured to transmit and/or receive sensor data. Throughout storage and/or transport, sensor data representative of the sensor signals may be recorded and/or transmitted, either continuously or when unexpected conditions occur (e.g., a temperature level signal falls outside predetermined thresholds. An automatic alert may be sent to the sender's (e.g., a field technician's) and/or the recipient's (e.g., a laboratory's) computer.

The sensor of the monitoring device may be incorporated into systems, methods, and devices of the present disclosure in different ways including, but not limited to, being inserted in, fastened to, and/or otherwise integrated with a storage and/or transport container; inserted in, fastened to, and/or otherwise integrated with a sample collection container; or even inserted in, fastened to, and/or otherwise integrated with a sample collection device like a swab (e.g., in the swab shaft).

In a preferred embodiment, a monitoring device is provided with the system to store and/or transmit temperature-related data. A monitoring device may utilize a range of effects to measure temperature levels such as chemical, electrical, and/or mechanical sensors. A monitoring device may include a mercury-in-glass thermometer, a Galileo thermometer, an alcohol thermometer, a liquid crystal thermometer, an infrared thermometer, a recording thermometer, a thermistor, a thermocouple, and/or another means of temperature/heat sensing.

In some embodiments, a monitoring device is provided with the system to store and/or transmit position or location-related data using any device that can determine its geographical location. For example, a monitoring device may include a global positioning system (GPS) receiver or a global navigation satellite system (GNSS) receiver. A GPS receiver may provide, for example, any standard format data stream, such as a National Marine Electronics Association (NMEA) data stream, or other data formats. In other embodiments, a monitoring device may include any device or mechanism that may determine location by any other means, such as performing triangulation by use of cellular radiotelephone towers. A variety of geographic location information may be requested by a processor and provided by a GPS module to the processor including, but not limited to, time (coordinated universal time-UTC), date, latitude, north/south indicator, longitude, east/west indicator, number and identification of satellites used in the position solution, number and identification of GPS satellites in view and their elevation, azimuth and SNR values, and dilution of precision values. Accordingly, it should be appreciated that in some embodiments a monitoring device may provide a wide variety of geographic information as well as timing information (e.g., one or more time stamps).

In further embodiments, a monitoring device is provided with the system to store and/or transmit pressure data, radiation data, and/or shock data. A monitoring device may utilize a range of effects to measure pressure levels, including converting pressure to some intermediate form such as displacement, which can then be converted into an electrical output such as voltage or current. A pressure sensor may include a strain gage transducer, a variable capacitance transducer, a piezoelectric transducer, and/or another means of pressure sensing. Likewise, a monitoring device may utilize a range of effects to measure levels (e.g., radioactivity, radiation exposure, and/or radiation absorption) of ionizing radiation and/or nonionizing radiation, such as electromagnetic radiation (including visible light). A radiation sensor may include a radiometer, a roentgenometer, a Geiger counter, a microR meter, a multichannel analyzer, an ionization chamber, a neutron REM meter, a radon detector, a liquid scintillation counter, a proportional counter, a film badge, a thermoluminescent dosimeter badge, an optically stimulated luminescence badge, and/or another means of radiation detection. A monitoring device may also utilize a range of effects to measure shock pulses and/or vibration levels, using, for example, a piezoelectric accelerometer, a piezoresistive accelerometer, and/or another means of shock sensing.

### Culture Media

According to some embodiments, as temperature is maintained to selectively promote (and/or inhibit) organism growth, organism viability, biochemical reactions, and/or chemical reactions, one or more of these goals may be furthered by inclusion of culture media, nutrients, reagents, or other substances. For example, culture media containing antibiotics may be added to a specimen container for selection of Gram-negative carbapenamase-resistant enterobacteriacae (CRE), such as Escherichia coli (E. coli). Culture media may also contain, for example, bile salts and/or some other Gram-positive bacterium inhibitor to increase the ratio of Gram negative CRE and/or Extended Spectrum Beta-lactamase (ESBL) bacteria during transport. Brain Heart Infusion (BHI) Broth and/or Tryptic Soy Broth (TSB) may be included to promote growth of mesophilic bacteria in a sample. In some embodiments, eukaryotic cells may be added to tissue cultures to promote growth of viruses. A substance may also be included to monitor organism growth, chemical reaction rates, levels of waste products, and/or other sample conditions including, but not limited to, pH indicators, radioactive isotopes, chemicals that change color in response to temperature change, chemicals for color-change biochemistry assays and analysis of optical absorption and emission spectra, and DNA probes that produce signal changes upon biological amplification.

### More Efficient Analysis, Diagnosis, Therapy, and Monitoring

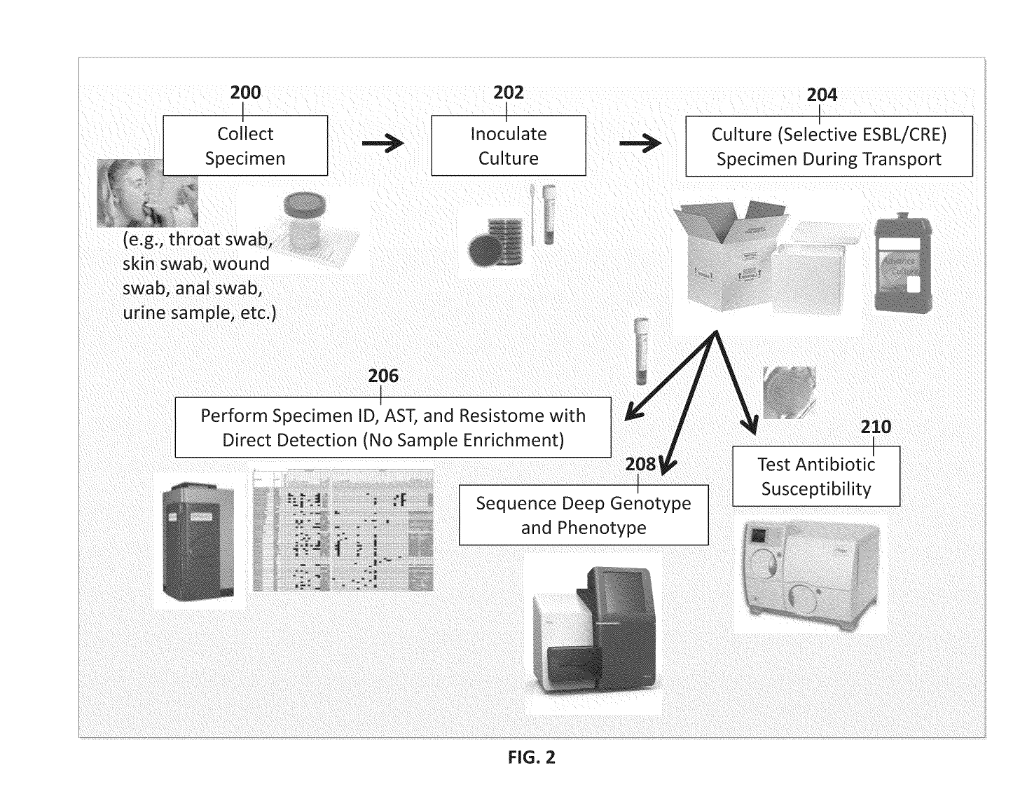

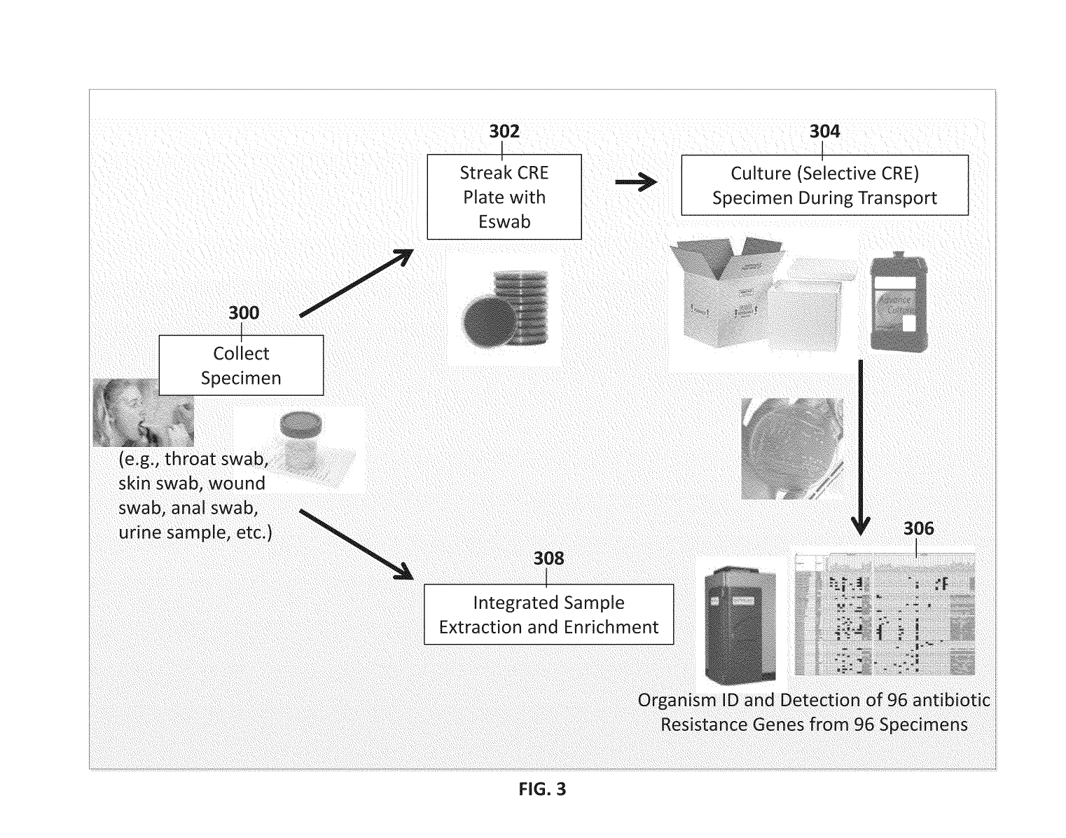

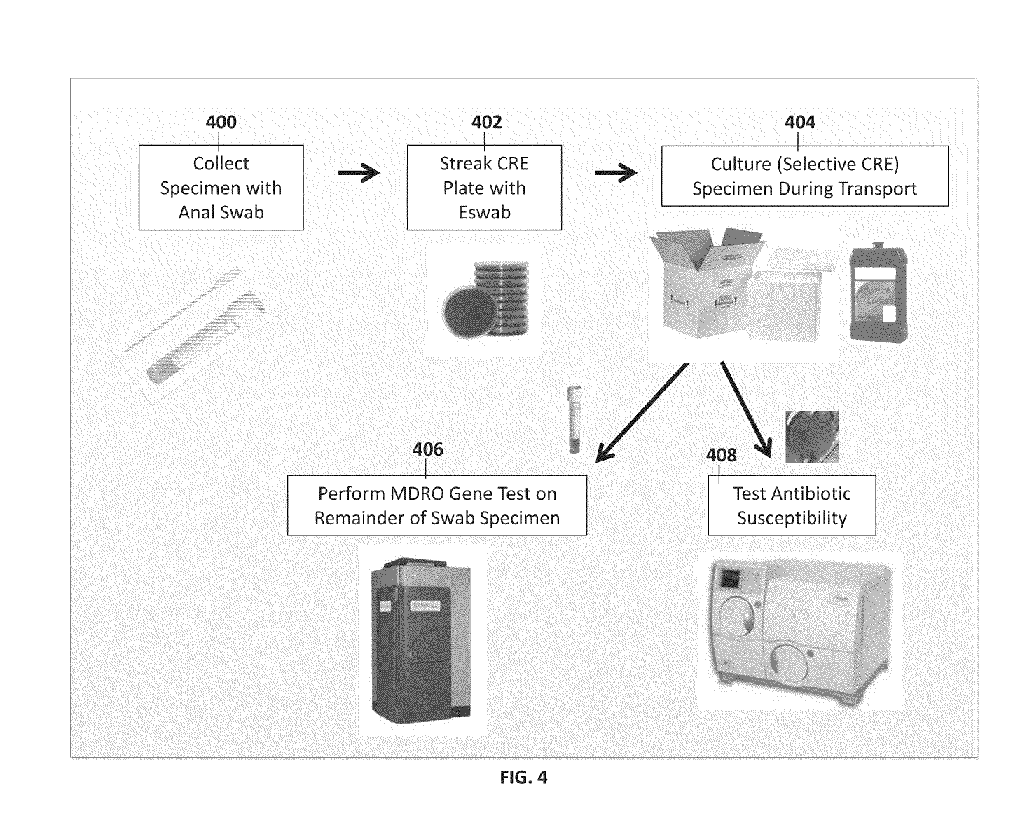

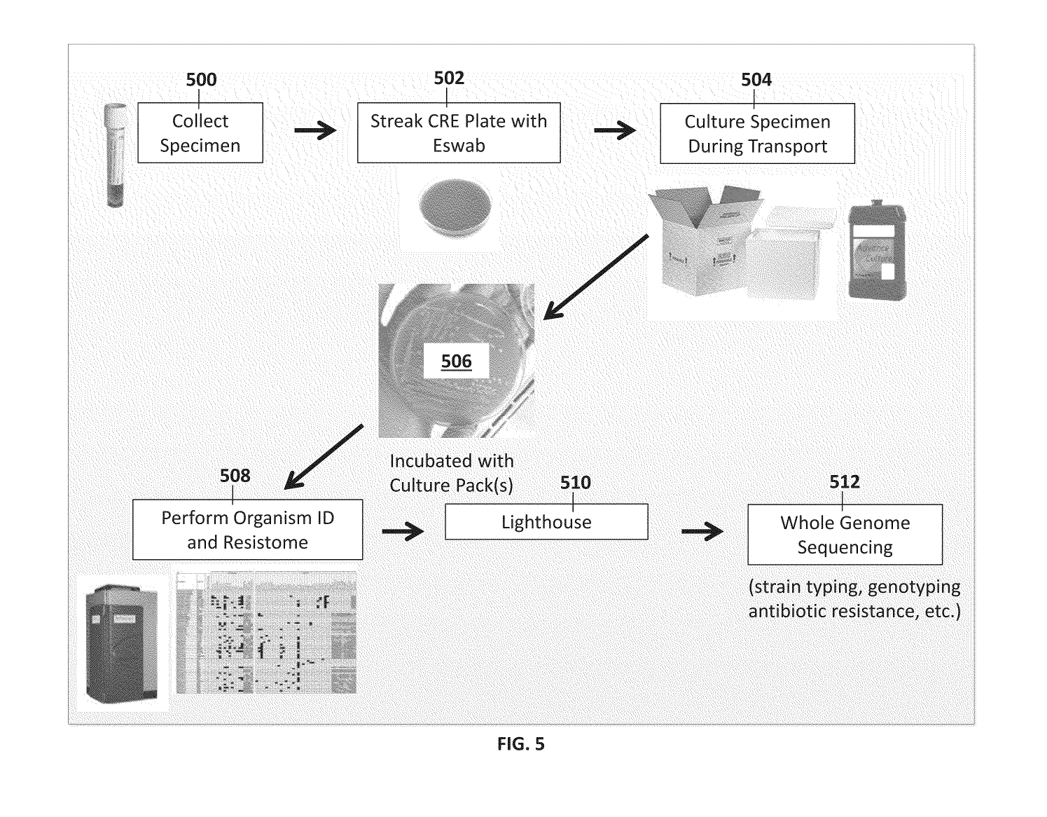

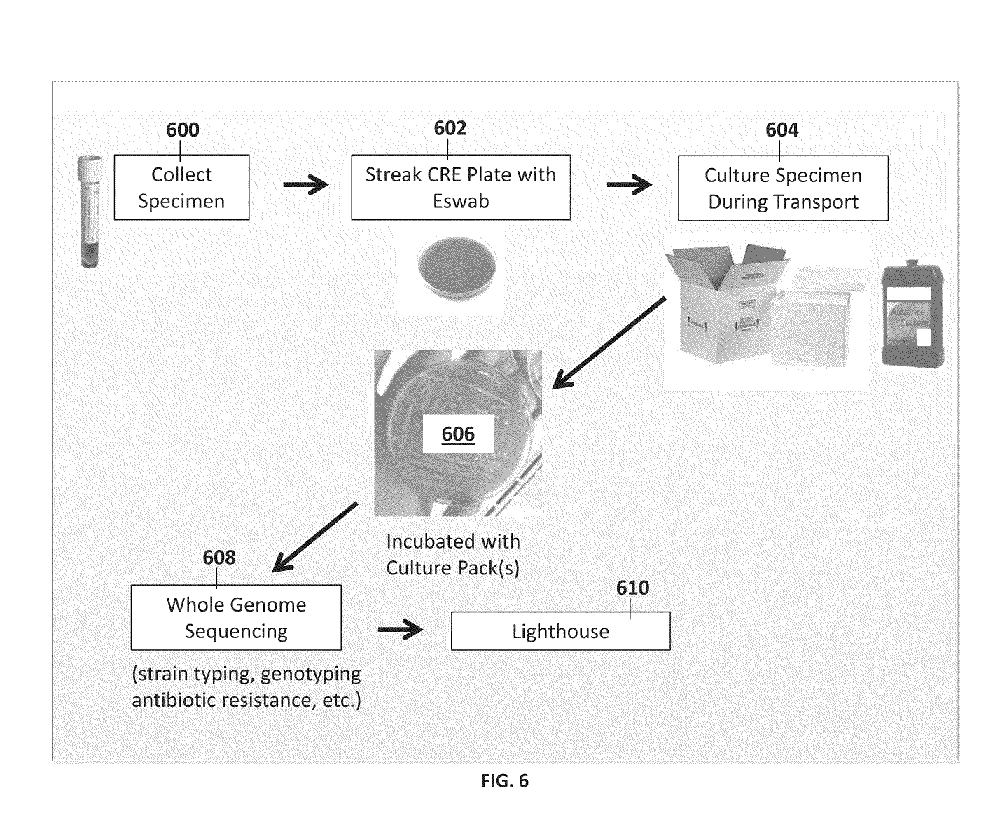

Delay can be a costly side-effect of storing and/or transporting samples. Once a laboratory receives a sample, further storage and/or time-consuming processing and extraction techniques may be required. However, by using storage and/or transport time to selectively promote (and/or inhibit) organism growth, organism viability, biochemical reactions, and/or chemical reactions, some embodiments minimize or at least utilize these delays. FIGS. 2-6 are flow diagrams illustrating processes for collecting and transporting samples for further testing and analysis in accordance with some embodiments.

According to some embodiments, the promotion (and/or inhibition) of organism growth, organism viability, biochemical reactions, and/or chemical reactions in a sample can be effective toward and/or increase the efficiency of analytic, diagnostic, therapeutic, and/or disease monitoring applications including, but not limited to, measurements and analysis of turbidity, oxygen consumption, carbon dioxide production, viscosity, and/or blood cultures; pH-dependent fluorescence or colorimetric changes; immunoassays; molecular diagnostics (PCR-based or non-PCR-based); and DNA sequencing, either as an extension of what might have been an isolate/pure sample or as a metagenomic study to identify more prominent genomes under different conditions.

The following examples further illustrate some embodiments.

### EXAMPLE 1

Two Nalgene® bottles (available from Nalge Nunc, Rochester, N.Y.), each containing 250 grams of 99% pure eicosane (available as Aldrich 219274-500G from Sigma-Aldrich (St. Louis, Mo.)), were warmed above 36° Celsius. The warmed bottles (and their contents) were placed in a carrier box comprising 3-inch thick walls of Styrofoam™ insulation (available from Dow Chemical Company (Marlborough, Mass.)) on all sides, top, and bottom, closed to form an internal storage volume of 5×3×3.5 inches.

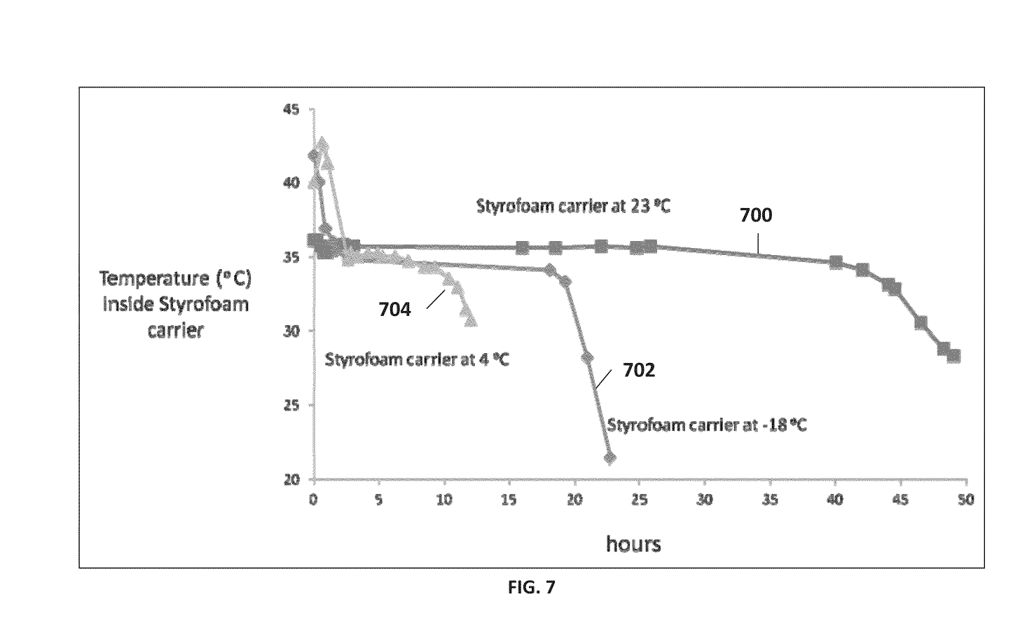

FIG. 7 is a plot of temperature inside a carrier for storing and/or transporting samples as a function of time in accordance with some embodiments. As shown in FIG. 7, the temperature of the internal storage volume was monitored as when the carrier box was stored at the following ambient temperatures: (i) room temperature, i.e., about 23° Celsius; (ii) about 4° Celsius, and (iii) about −18° Celsius. When the carrier box was stored at (i) room temperature, the temperature 700 of the internal storage volume was maintained at about 36° Celsius for approximately 35 hours. When the carrier box was stored at (ii) about 4° Celsius, the temperature 702 of the internal storage volume was maintained at about 36° Celsius for approximately 16 hours. When the carrier box was stored at (iii) about −18° Celsius, the temperature 704 of the internal storage volume was maintained at about 36° Celsius for approximately 6 hours.

### EXAMPLE 2

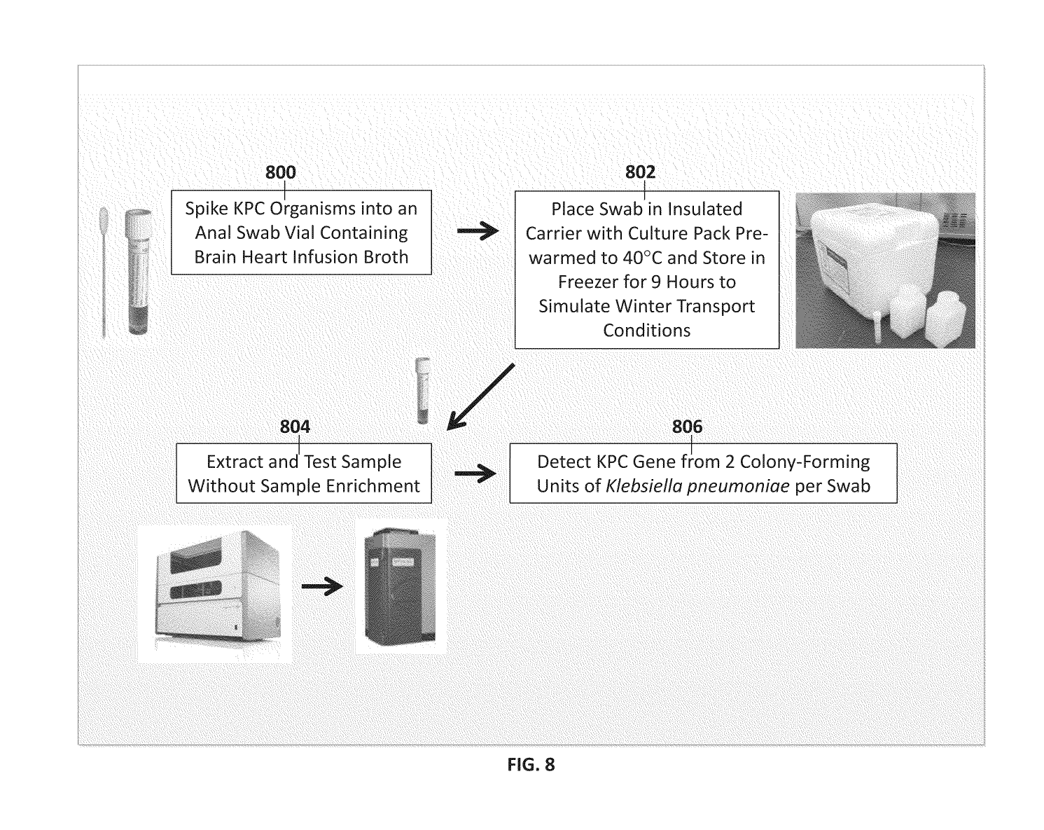

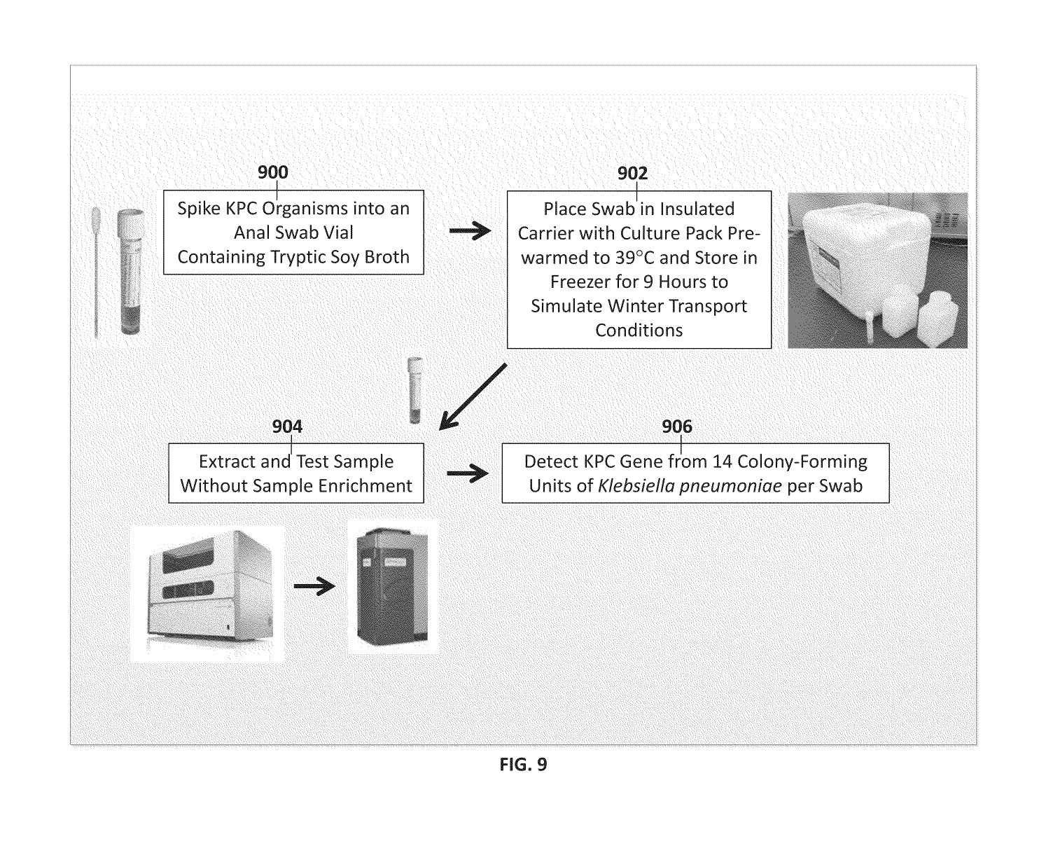

FIGS. 8 and 9 are flow diagrams illustrating processes for incubating specimen culture plates for further testing and analysis in accordance with some embodiments. Anal swabs were collected from healthy human volunteer subjects using BD ESwabs, which are flocked applicator swabs stored in polypropylene screw-cap tube filled with 1 mL of modified Liquid Amies Medium (available as BD 220245 from BD Diagnostics (Sparks, Md.)). For the method shown in FIG. 8, the modified Liquid Amies Medium was replaced with 1 mL of Brain Heart Infusion (BHI) Broth (available as B9993 from Teknova (Hollister, Calif.)). For the method shown in FIG. 9, the media was instead replaced with Tryptic Soy Broth (TSB) (available as Remel™ R07222 from Thermo Fisher Scientific (Lenexa, Kans.)). Clean swabs were prepared in a similar manner as control samples.

As in steps 800 and 900, the resultant swab samples were spiked with indicated levels of Klebsiella pneumoniae, a Gram-negative bacterium that harbors the antibiotic resistance gene KPC. Spiked levels of K. pneumoniae were determined through parallel counting of colony forming units (CFUs) on blood agar plates (BD BBL™ Trypticase™ Soy Agar with 5% Sheep Blood (TSA II™) (20/sp) or BD BBL™ Trypticase™ Soy Agar with 5% Sheep Blood (TSA II™) (100/sp), available as BD 221239 or 221261 from BD Diagnostics (Sparks, Md.)). Half of a BBL™ Sensi-Disc™ Antimicrobial Susceptibility Test Disc with 30/10 mcg ceftazidime/calvulanic acid (available as BD 231753 from BD Diagnostics (Sparks, Md.)) was added to some of the swab samples in the method illustrated by FIG. 8, as indicated in Table 2.

The swab samples were placed either in a commercial incubator at about 37° Celsius or in the Styrofoam™ carrier from Example 1, as in steps 802 and 902, with two Nalgene® bottles, each containing 250 grams of 99% pure eicosane, that were pre-warmed to about 40° Celsius or 39° Celsius. The Styrofoam™ carrier was stored in a freezer at about −18° Celsius to simulate winter transport conditions. All swab samples were incubated for about 9 hours.

Following incubation, in steps 804 and 904, 500 μL of the BHI Broth from each swab sample underwent automated extraction of bacterial DNA using the MagNA Pure™ 96 Instrument (available from Roche Diagnostics (Indianapolis, Ind.)), which rendered 100 μL of extracted DNA. The extracted DNA samples were analyzed in steps 806 and 906, using real-time PCR and detection for the KPC gene using the BioMark™ HD System with the 192.24 Dynamic Array™ Integrated Fluidic Circuit (IFC), a microfluidic array capable of analyzing 192 samples with 24 PCR assays (both available from Fluidigm (South San Francisco, Calif.)). The number of initially spiked K. pneumoniae genomes per PCR without culture growth is vanishingly small as indicated in the tables below because only 2.6 nL from each 100 μL sample of extracted DNA were analyzed by PCR using the BioMark™ HD System.

As shown in Table 2, for the method illustrated in FIG. 8, as few as 2 to 139 initially spiked K. pneumoniae genomes were detected per clean swab sample after storing the clean swab samples without antibiotic in the Styrofoam™ carrier at −18° Celsius for 9 hours. This matches the sensitivity for the spiked anal swab samples without antibiotic that were incubated for 9 hours in a commercial incubator at 37° Celsius, as shown in Table 3. As shown in Table 2, as few as 139 initially spiked K. pneumoniae genomes per anal swab sample were detected after storing the anal swab samples with antibiotic in the Styrofoam™ carrier at −18° Celsius for 9 hours.

TABLE 2

| | Number of | | | | |
|---|---|---|---|---|---|
| Number of K. | Spiked K. | | | | |
| pneumoniae | pneumoniae | Incubation in Styrofoam ™ Carrier | | | |
| Colony | Genomes | Clean Swab without | | Anal Swab | |
| Forming | per PCR | Antibiotic | | with Antibiotic | |
| Units (CFUs) | Without | | Positive | | Positive |
| Spiked per | Culture | PCR | PCR | PCR | PCR |
| Swab | Growth | Result | Cycle # | Result | Cycle # |
| >300 | 3.9E−03 | positive | 11 | positive | 22 |
| 139 | 1.8E−04 | positive | 12 | positive | 14 |
| 7 | 9.0E−05 | positive | 14 | negative | negative |
| 2 | 2.6E−05 | positive | 18 | negative | negative |
| 0 | 0 | negative | negative | negative | negative |
| neg. control | | negative | negative | negative | negative |

TABLE 3

| | Number of Spiked | | |
|---|---|---|---|
| Number of K. | K. pneumoniae | Incubation in Incubator at 37° C. | |
| pneumoniae | Genomes per PCR | Anal Swab without Antibiotic | |
| CFUs Spiked per | Without Culture | PCR | |
| Swab | Growth | Result | Positive PCR Cycle # |
| >300 | 3.9E−03 | positive | 11 |
| 139 | 1.8E−03 | positive | 11 |
| 7 | 9.0E−05 | positive | 13 |
| 2 | 2.6E−05 | positive | 22 |
| 0 | 0 | negative | negative |
| neg. control | | negative | negative |

As shown in Table 4, for the method illustrated in FIG. 9, as few as 1 to 91 initially spiked K. pneumoniae genomes and as few as 62 initially spiked vancomycin enterocci (VRE), specifically Van-A enterocci (resistant to vancomycin and teicoplanin), were detected per clean swab sample after storing the clean swab samples without antibiotic in the Styrofoam™ carrier at −18° Celsius for 9 hours.

TABLE 4

| | | Van-A Enterococci (Resistant to | |
|---|---|---|---|
| K. pneumoniae | | Vancomycin and Teicoplanin) | |
| Number of CFUs | Positive PCR | Number of CFUs | Positive PCR |
| Spiked per Swab | Cycle # | Spiked per Swab | Cycle # |
| >300 | 13 | >300 | 23 |
| 91 | 14 | 62 | 22 |
| 14 | 17 | 11 | negative |
| 1 | 22 | 0 | negative |
| 0 | negative | 0 | negative |

### EXAMPLE 3

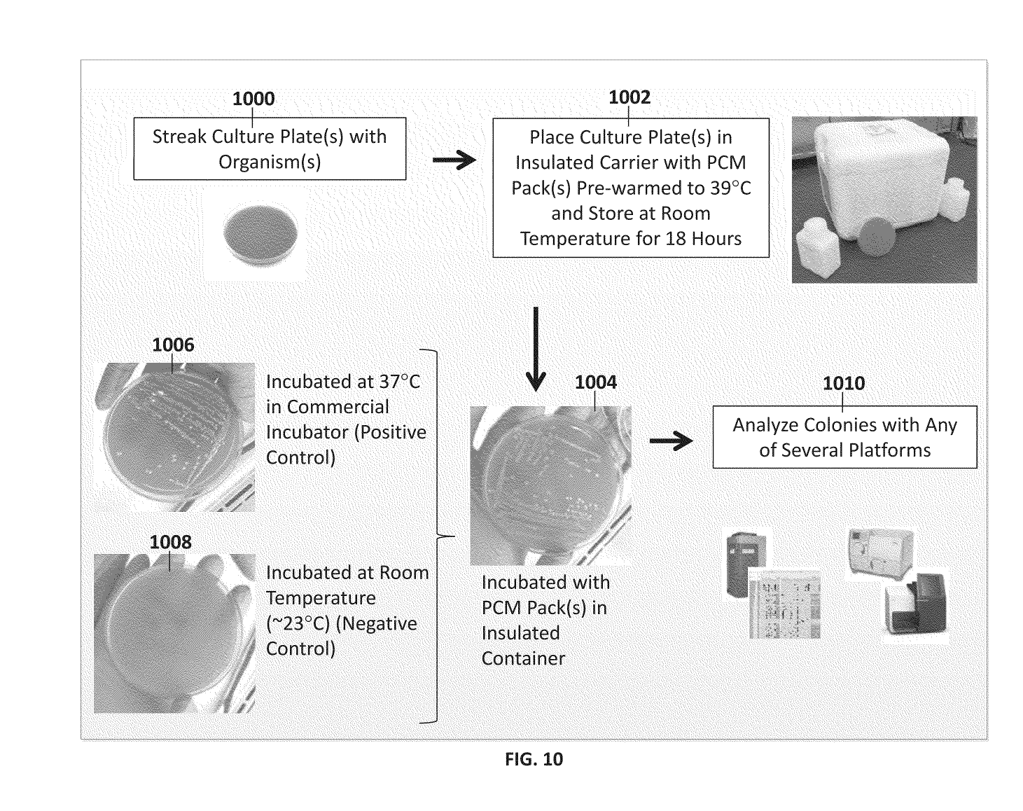

FIG. 10 is a flow diagram illustrating processes for incubating specimen culture plates for further testing and analysis in accordance with some embodiments. In step 1000, a glycerol stock of K. pneumoniae was streaked for isolation on blood agar plates (BD BBL™ Trypticase™ Soy Agar with 5% Sheep Blood (TSA II™) (20/sp) or BD BBL™ Trypticase™ Soy Agar with 5% Sheep Blood (TSA II™) (100/sp), available as 221239 or 221261 from BD Diagnostics (Sparks, Md.)). In step 1002, one blood agar plate was placed in a Styrofoam™ carrier similar to that described in Example 1, with two Nalgene® bottles, each containing 250 grams of 99% pure eicosane, that were pre-warmed to about 39° Celsius. Meanwhile, a second blood agar plate (i.e., the positive control) was placed in a commercial incubator at about 37° Celsius. A third blood agar plate (i.e., the negative control) was stored at room temperature (i.e., about 23° Celsius). The three blood agar plates were incubated 18 hours.

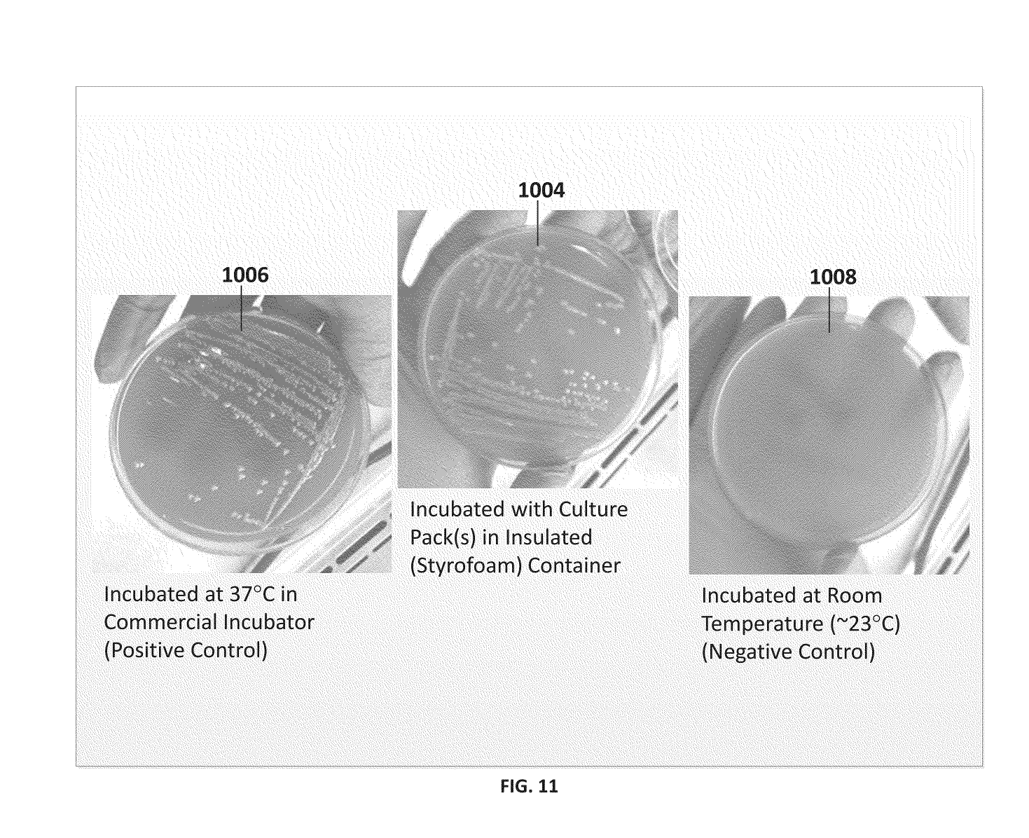

As shown in FIGS. 10 and 11, a series of photographs illustrate specimen culture plates after an incubation period in accordance with some embodiments. The blood agar plate from the Styrofoam™ carrier 1004 exhibited bacterial growth similar to the positive control 1006. In contrast, bacterial growth was absent on the negative control 1008. As in step 1010 of FIG. 10, K. pneumoniae colonies in the blood agar plate from the Styrofoam™ carrier 1004 could then be further analyzed using any of several platforms.

### EXAMPLE 4

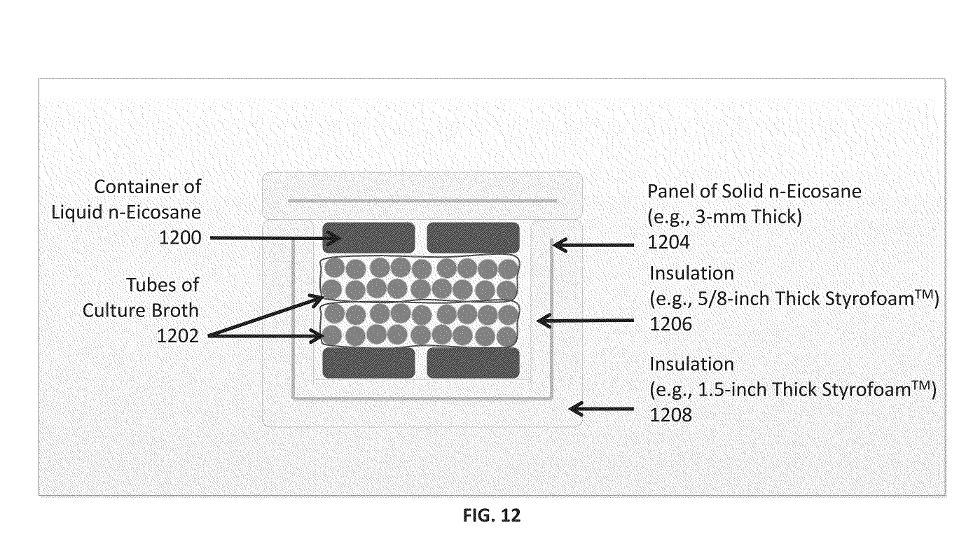

FIG. 12 is a diagram illustrating a binary carrier for storing and/or transporting a sample in accordance with some embodiments. The binary carrier is designed to maintain the contents of the carrier at approximately 36° Celsius under lower and higher ambient storage temperatures. Liquid n-Eicosane (e.g., one or more containers) inside the storage compartment of the carrier maintains contents at approximately 36° Celsius when the carrier is stored at ambient temperatures below 36° Celsius (as liquid n-Eicosane becomes solid). In one embodiment, as shown in FIG. 12, four plastic containers 1200, each containing approximately 320 grams of 99% pure n-Eicosane (available as Aldrich 219274-500G from Sigma-Aldrich (St. Louis, Mo.)), were warmed above 36° Celsius and placed in the carrier storage compartment. Thirty-six plastic tubes 1202 were distributed inside two plastic transportation bags and served as representative carrier content. Each plastic tube 1202 contained approximately 2 mL of liquid culture broth at room temperature.

According to some embodiments, solid n-Eicosane may be attached to or incorporated within one or more walls of the carrier to further protect the carrier content against ambient temperatures greater than 36° Celsius (as solid n-Eicosane becomes liquid). In one embodiment, as shown in FIG. 12, six plastic panels 1204, each containing a 3-mm thickness of solid technical grade n-Eicosane (available as CAS #112-95-8 from City Chemical LLC (West Haven, Conn.)), were placed against the internal sides, bottom, and top of the carrier storage compartment. Each panel of solid n-Eicosane was held in place between a first layer of insulation 1206, such as an insulated carrier with 1.5-inch thick walls of Styrofoam™ insulation (available from Dow Chemical Company (Marlborough, Mass.)), and a second layer of insulation 1208, such as a ⅝-inch thick panels of Styrofoam™ insulation.

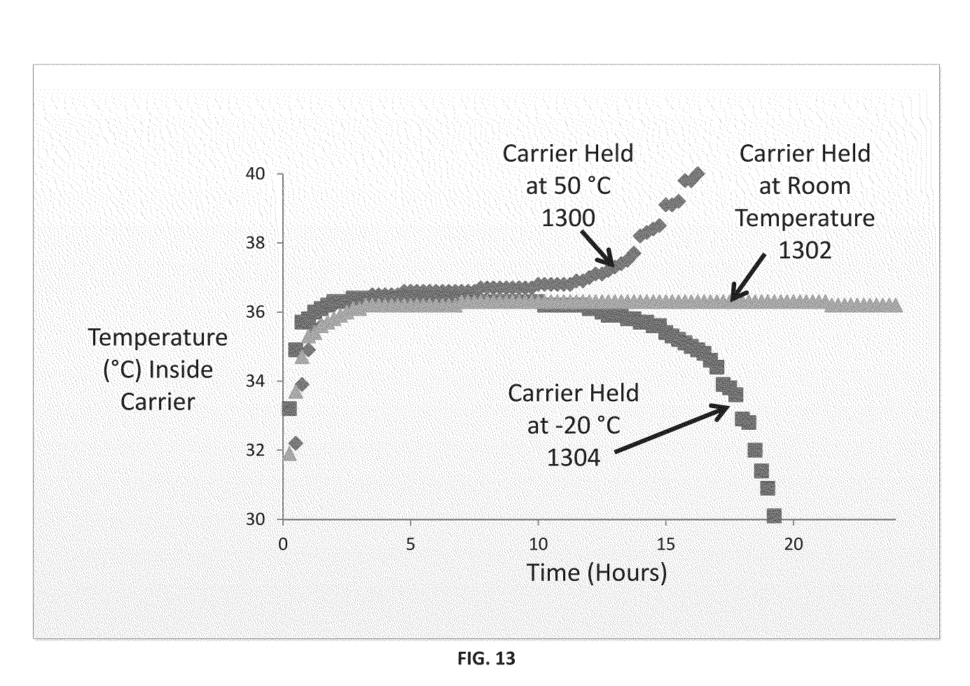

FIG. 13 is a plot of temperature inside the carrier shown in FIG. 12 as a function of time in accordance with some embodiments. The temperature of the carrier contents was monitored as the carrier was stored at the following ambient temperatures: (i) about 50° Celsius 1300; (ii) room temperature or about 23° Celsius 1302; and (iii) about −20° Celsius 1304. When the carrier was stored at (iii) about 50° Celsius 1300, the temperature of the contents was maintained at about 36° Celsius for approximately 10 hours. When the carrier was stored at (i) room temperature 1302, the temperature of the contents was maintained at about 36° Celsius for more than 48 hours. When the carrier was stored at (ii) about −20° Celsius 1304, the temperature of the contents was maintained at about 36° Celsius for approximately 14 hours.

### EXAMPLE 5

According to some embodiments, a sample may be grown during storage and/or transport. For example, antibiotic-resistant bacteria were grown in broth culture during a shipment in accordance with some embodiments. Anal swabs were collected from healthy human volunteer subjects and placed in screw-cap tubes with 2 mL of TSB (Remel™ R07222 from Thermo Fisher Scientific (Lenexa, Kans.)) and 3 μg per mL of the antibiotic Ceftriaxone (available as BBL™ Sensi-Disc™ Susceptibility Test Discs 231635 from Becton, Dickinson and Company (Franklin Lakes, N.J.)).

Swab/broth samples were spiked with indicated levels of K. pneumoniae harboring the antibiotic resistance gene KPC, where spiked levels of K. pneumoniae were determined through parallel counting of CFUs on blood agar plates (TSA II™). Swab/broth samples were placed either in a laboratory incubator at 37° Celsius (positive control), a laboratory refrigerator at 4° Celsius (negative control), or an insulated (Styrofoam™) shipping box with four 8-ounce bottles (Nalgene®), each bottle containing 250 grams of 99% pure n-Eicosane pre-warmed at about 40° Celsius as in the methods illustrated by FIGS. 8 and 9. The shipping box was labeled in compliance with U.S. regulations for transport of Biological Substances, Category B (infectious substances that are not in a form generally capable of causing permanent disability or life-threatening or fatal disease in otherwise healthy humans or animals when exposure to it occurs, including substances transported for diagnostic or investigational purposes).

FedEx® delivery service personnel picked up the shipping box from a laboratory and returned the shipping box back to the laboratory about 24 hours later. Upon receipt, 500 μL of broth from each cultured swab/broth sample underwent automated extraction of bacterial DNA using the MagNA Pure™ 96 Instrument. Extracted DNA samples (about 100 μL) were analyzed in triplicate by microfluidic real-time PCR tests on the BioMark™ HD System with the 192.24 Dynamic Array™ IFC. The number of initially spiked K. pneumoniae organisms per PCR without culture growth was extremely small because only 2.6 nL from the 100 μL of extracted DNA are used per PCR on the 192.24 Dynamic Array™ IFC.

As shown below in TABLE 5, samples spiked with K. pneumoniae were positive for the KPC gene even down to two CFUs of K. pneumonia per swab/broth sample after being stored 24 hours in the laboratory incubator at 37° Celsius (positive control) or shipped with n-Eicosane as shown in TABLE 5. A consistent PCR Ct value of 9 was observed for these culture samples because they reached stationary phase after 24 hours of bacterial growth. In contrast, the KPC gene was detected with high PCR Ct values only at higher levels of spiked K. pneumonia for samples incubated in the refrigerator due to much less culture growth. Thus, pre-warmed n-Eicosane supported growth of antibiotic-resistant bacteria in broth culture during shipment in accordance with some embodiments.

TABLE 5

| | | | Samples Stored in Lab | | Samples Stored in Lab | |
|---|---|---|---|---|---|---|
| | Samples Shipped with | | Incubator at 37° C. | | Incubator at 37° C. | |
| | n-Eicosane | | (positive control) | | (positive control) | |
| Number of | Acuitas ® | | Acuitas ® | | Acuitas ® | |
| K. | MDRO | | MDRO | | MDRO | |
| pneumoniae | Gene Test | | Gene Test | | Gene Test | |
| CFUs per | Result for | PCR Ct | Result for | PCR Ct | Result for | PCR Ct |
| Sample | KPC Gene | Value | KPC Gene | Value | KPC Gene | Value |
| >300 | positive | 9 | positive | 9 | positive | 17 |
| >300 | positive | 9 | positive | 9 | positive | 20 |
| >300 | positive | 9 | positive | 9 | positive | 24 |
| 100 | positive | 9 | positive | 9 | negative | |
| 10 | positive | 9 | positive | 9 | negative | |
| 2 | positive | 9 | positive | 9 | negative | |
| 0 | negative | | negative | | negative | |
| 0 | negative | | negative | | negative | |
| neg. | negative | | negative | | negative | |
| control | | | | | | |

### Conclusion

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The above-described embodiments can be implemented in any of numerous ways. For example, embodiments of designing, constructing, and monitoring the systems, apparatus, and methods disclosed herein may be implemented using hardware, software or a combination thereof. When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

The various methods or processes (e.g., of designing and making the retention/delivery structure disclosed above) outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All publications, patent applications, patents, and other references mentioned herein are incorporated by reference in their entirety, including the International Patent Application filed on Aug. 25, 2015, and entitled “Systems, Methods, and Devices for Temperature Control,” which also claims a priority benefit of U.S. Provisional Patent Application No. 62/041,405, filed on Aug. 25, 2014, and entitled “Systems, Methods, and Devices for Temperature Control.”

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, the terms “about,” “approximately,” and the like are to be understood to mean +/−10% of the total amount stated, e.g., about 5 would include 4.5 to 5.5, about 10 would include 9 to 11, and about 100 would include 90-110.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A system comprising:
at least one pack having sealed therein at least one phase change material selected and configured to maintain a predetermined temperature range between about 22° Celsius and about 100° Celsius during a predetermined time period; and
a structure configured to receive and retain the at least one pack.

2. The system of claim 1, wherein the system maintains at least one sample within the predetermined temperature range during the predetermined time period.

3. The system of claim 2, wherein the system promotes at least one of organism growth, organism viability, a biochemical reaction, and a chemical reaction in the at least one sample during the predetermined time period.

4. The system of claim 2, wherein the system inhibits at least one of organism growth, organism viability, a biochemical reaction, and a chemical reaction in the at least one sample during the predetermined time period.

5. The system of claim 2, wherein the system is further configured such that at least one of pre-analytical processing, analytical testing, medical diagnostic testing, and medical therapy is applied to the at least one sample during the predetermined time period.

6. The system of claim 1, wherein the at least one pack is pre-heated to an initial temperature, the initial temperature being approximately the same as a phase change temperature of one of the at least one phase change material.

7. The system of claim 6, wherein the initial temperature is the phase change temperature +/−2° Celsius.

8. The system of claim 6, wherein the phase change temperature is the melting temperature of the one of the at least one phase change material.

9. The system of claim 6, wherein the initial temperature is about 40° Celsius.

10. The system of claim 1, further comprising a monitoring device including at least one sensor.

11. The system of claim 10, wherein the at least one sensor comprises at least one of a thermometer, a thermistor, a thermocouple, a global positioning system (GPS) receiver, a global navigation satellite system (GNSS) receiver, a transducer, a radiometer, a dosimeter, and an accelerometer.

12. The system of claim 1, wherein the at least one phase change material comprises at least one of a paraffin, a fatty acid, a salt hydrate, a eutectic composition, a cross-linked polyethylene, and a polyalcohol.

13. The system of claim 1, wherein the at least one phase change material comprises n-Eicosane.

14. The system of claim 13, wherein the n-Eicosane is initially in a substantially liquid phase.

15. The system of claim 1, wherein the at least one phase change material comprises heneicosane.

16. The system of claim 15, wherein the heneicosane is initially in a substantially solid phase.

17. A system comprising:
a structure comprising at least one phase change material selected and configured to maintain a predetermined temperature range between about 22° Celsius and about 100° Celsius during a predetermined time period.

18. The system of claim 17, wherein the at least one phase change material is encapsulated in at least one compartment integrated into the structure.

19. The system of claim 17, wherein the at least one phase change material is a thermal composite integrated into the structure.

20. A method for controlling temperature, comprising:
disposing, within a structure, at least one pack having sealed therein at least one phase change material selected and configured to maintain a predetermined temperature range between about 22° Celsius and about 100° Celsius during a predetermined time period such that the predetermined temperature range is maintained during the predetermined time period.
